(19) United States
(12) Patent Application Publication
(10) Pub. No.: US 2023/0386074 A1
(43) Pub. Date: Nov. 30, 2023

(54) COMPUTER VISION AND MACHINE LEARNING TO TRACK SURGICAL TOOLS THROUGH A USE CYCLE

(71) Applicants: Stephen CANTON, Pittsburgh, PA (US); Dukens LABAZE, Pittsburgh, PA (US)

(72) Inventors: Stephen CANTON, Pittsburgh, PA (US); Dukens LABAZE, Pittsburgh, PA (US)

(21) Appl. No.: 18/032,753

(22) PCT Filed: Oct. 19, 2021
(86) PCT No.: PCT/US2021/055678

§ 371 Date: Apr. 19, 2023

Related U.S. Application Data
(60) Provisional application No. 63/093,394, filed on Oct. 19, 2020.

(51) Int. Cl. G06T 7/73; G06V 10/764; A61B 90/90; G16H 40/67; G16H 40/20

(52) U.S. Cl. CPC G06T 7/73; G06V 10/764; A61B 90/90; G16H 40/67; G16H 40/20; G06T 2207/30004; G06T 2207/30204; G06T 2207/30232

(57) ABSTRACT

Disclosed herein are systems and methods for organizing and tracking sterilizable tools and consumables through an entire use cycle in a perioperative environment, such as through the multiple steps of the use, decontamination, sterilization, assembly, and distribution workflow. Also disclosed are systems and methods for optimizing the collections of sterilizable tools and consumables provided for use in specific procedures or by specific members of the perioperative environment. The methods generally include using computer vision to capture images of the surgical tools along multiple steps of the use cycle, and machine learning algorithms to process the images and determine an identity of each surgical tool. This information may be compared against a list of expected surgical tools to determine if any are missing and/or if the correct collection is provided.

### CROSS REFERENCE TO RELATED APPLICATIONS

The present application claims the benefit of priority under 35 U.S.C. § 119(e) of prior U.S. Provisional Application Ser. No. 63/093,394, filed Oct. 19, 2020, the entire content of which is incorporated herein.

### TECHNICAL FIELD

This disclosure pertains generally to systems and methods for tracking surgical tools in real time through a complete use cycle. The disclosure further pertains to methods for optimizing provision of surgical tool sets for specific procedures and/or personnel.

### BACKGROUND

The sterile processing department (SPD) is an integrated area in hospitals and other health care facilities that performs cleaning, decontamination, sterilization, and other processing on surgical tools such as medical devices, surgical instruments and equipment, and consumables. These processes are required for subsequent use of the surgical tools by healthcare workers in operating rooms of the hospital and for other aseptic procedures, e.g., catheterization, wound stitching and bandaging in a medical, surgical, maternity, or pediatric ward.

The SPD, central supply department (CSD), and operating room (OR) are all considered part of the perioperative environment. Typically, multiple people participate in the perioperative environment and its related procedures. For example, participants during an operation can include a chief surgeon, an assistant surgeon, an anesthesiologist, a scrub nurse, and a circulating nurse. Within the SPD and CSD and the related workflows that are being conducted, further technicians are involved. Overall, there are many personnel in the perioperative environment, all of whom may interact with the surgical tools used in a procedure.

Intensive efforts are invested in keeping track of all surgical tools and disposables to make sure that no tool is unintentionally left inside the patient's body, and that no surgical tool is incorrectly packed and/or processed prior to use. Moreover, different procedures and different surgeons require different collations of surgical tools, i.e., toolkits and/or surgical trays, making organization and tracking of the tools an even larger task.

Thus, careful monitoring of workflows and processes relating to all surgical tools are performed in the perioperative environment, including counting of all surgical tools before, during, and after an operation. Currently, tracking and recording the usages of surgical tools involves the surgical staff manually performing these tasks using a computer, by making a note on a whiteboard, or via verbal readback. Such counting is a tedious job that requires resources. Moreover, counting the surgical tools towards the end of an operation increases both the time the patient's body is open with the associated risks, anesthesia time, and the down-time of the operating room. In addition, counting is not always error-free, with the risk that the wrong surgical tools are supplied to an OR, or that surgical tools are left within the patient's body.

Accordingly, systems and methods for tracking surgical tools and their usage, such as in real time and/or through a complete use cycle, are desired and an object of the present disclosure.

### SUMMARY

The present disclosure provides systems and methods for organizing and tracking sterilizable tools and consumables through an entire use cycle, such as through the multiple steps of the use, decontamination, assembly (e.g., assembly into trays or sets specific for procedures or storage), sterilization, and distribution workflow.

The present disclosure further provides systems and methods for optimizing collections of sterilizable tools and consumables provided for aseptic procedures in the perioperative environment.

Accordingly, the methods of the present disclosure generally comprise capturing one or more images of a plurality of surgical tools; processing the one or more images to determine identities of each surgical tool in the plurality of surgical tools; and recording the determined identities of each surgical tool. The methods may further comprise comparing the recorded identities of each surgical tool to an expected list of identities of the plurality of surgical tools, wherein the expected list may be determined based on a user input value or a fiducial marker found in the one or more images captured by the camera, such as a 2D or 3D code.

According to certain aspects of the method, the step of processing the one or more images to determine identities of each surgical tool in the plurality of surgical tools may comprise pattern recognition of a shape and size of each surgical tool and/or a tag attached to each surgical tool, such as a 2D or 3D code. Moreover, the steps of capturing, processing, and recording may be completed substantially continuously, such as in an operating room or as the plurality of surgical tools are transported throughout a perioperative environment. The steps of capturing, processing, and recording may be completed at specific locations within the perioperative environment, such as at least before an aseptic procedure in an operating room, after an aseptic procedure in the operating room, on arrival at a decontamination area, and during assembly into trays before sterilization. Based on the comparing step, the method may determine one or more surgical tools missing from the plurality of surgical tools, one or more surgical tools added to the plurality of surgical tools (e.g., extra tools not needed in the plurality of surgical tools), or a combination thereof.

According to certain aspects, the method may determine one or more surgical tools missing from the plurality of surgical tools based on the comparing step, and may provide an alert, such as on a computer screen or on an augmented reality display. The alert may include information comprising an identity of each of the one or more missing surgical tools. The alert may be provided as an overlay on the plurality of surgical tools (i.e., when the plurality of surgical tools are in a field of view). When provided on the augmented reality display, the information may be displayed in the field of view of a user, such as on a head mounted transmission display.

According to certain aspects of the method, the step of capturing an image of the plurality of surgical tools comprises capturing an image of an assembled tray comprising the plurality of surgical tools. As such, the method may further comprise creating an augmented reality information that relates to one or more missing surgical tools from the assembled tray by evaluating, via a machine learning algorithm, the image of the assembled tray captured by the camera; and outputting, the augmented reality information such that, in response to a user placing the assembled tray in a field of view of the user, an identity and/or correct location of the one or more missing surgical tools is perceivable in the field of view of the user. The identity and/or correct location of the one or more missing surgical tools may be overlaid on the assembled tray, wherein the identity and/or coy ed. location of the one or more missing surgical tools is based on a surgeon preference list and/or a procedure type. Those surgical tools on the list determined by the machine learning algorithm may be identified by a use rate of individual surgical tools in the plurality of surgical tools during a procedure in an aseptic environment. In this way, the method also enable optimization of surgical tools provided in a plurality of surgical tools by omitting those tools found not to be used in a procedure or by a surgeon.

The present disclosure also relates to methods for tracking a plurality of surgical tools through a use cycle, wherein the method comprises capturing images of the plurality of surgical tools using computer vision; processing the images to determine an identity of each of the surgical tools in the plurality of surgical tools; comparing the determined identities of each surgical tool to an expected list of identities of the plurality of surgical tools; based on the comparing step, determining if one or more surgical tools are missing from the plurality of surgical tools; creating an augmented reality information comprising an identity of each of the one or more missing surgical tools; and outputting the augmented reality information in a field of view of a user. The steps of capturing and processing may be completed substantially continuously as the plurality of surgical tools are transported throughout a perioperative environment. Moreover, the augmented reality information may be provided by a display unit, such as a head mounted transmissive display.

According to certain aspects of the method, the step of creating the augmented reality information may comprise evaluating, via a machine learning algorithm, the captured images; and outputting the augmented reality information such that, in response to a user placing the plurality of surgical tools in a field of view of the user, the identity and/or correct location of the one or more missing surgical tools is perceivable in the field of view of the user. The identity and/or correct location of the one or more missing surgical tools may be based on a surgeon preference list and/or a procedure type, wherein the surgeon preference list includes a list of surgical tools selected by a surgeon, or a list of surgical tools determined by the machine learning algorithm. According to certain aspects, the list of surgical tools determined by the machine learning algorithm is identified by a use rate of individual surgical tools in the plurality of surgical tools during a procedure in an aseptic environment.

The present disclosure also relates to non-transitory computer readable recording medium recorded with a program for causing a computer to track a plurality of surgical tools through a use cycle and/or with a program for optimizing tool usage in a perioperative environment according to any of the methods disclosed herein.

Moreover, the present disclosure further relates to a system for tracking a plurality of surgical tools through a use cycle and/or a system for optimizing tool usage in a perioperative environinent, wherein the system(s) comprise a processor; an optical imaging device coupled to the processor; and a memory coupled to the processor and storing processor-readable instructions that, when executed, cause the processor to perform any one or more of the methods disclosed herein.

### BRIEF DESCRIPTION OF THE DRAWINGS

A better understanding of the present disclosure will be had upon reference to the following detailed description when read in conjunction with the accompanying drawings, wherein like numerals represent like features in the various views. For clarity, only those elements which are useful to the understanding of the described embodiments have been shown and are detailed. It is to be noted that features and components in these drawings, illustrating views of embodiments of the present invention, unless stated to be otherwise, are not necessarily drawn to scale.

FIG. 1A shows an exemplary step in the sterilization, processing, and distribution pathway for surgical tools.

FIG. 1B shows another exemplary step in the sterilization, processing, and distribution pathway for surgical tools.

FIG. 1C shows an exemplary code positioned on a wrapped surgical tray according to the present disclosure.

FIG. 1D shows visualization of a location of a missing surgical tool according to methods of the present disclosure.

FIG. 2 shows an exemplary camera location in an operating room for use in systems and methods according to the present disclosure.

FIG. 3 shows exemplary camera locations in decontamination and assembly areas for use in systems and methods according to the present disclosure.

FIG. 4 is a flow chart depicting steps for removal of surgical tools from an operating room post-surgery.

FIG. 5 is a flow chart depicting steps for intake and cleaning of non-sterile surgical tools, such as after use during a procedure.

FIG. 6 is a flow chart depicting steps for assembly of cleaned surgical tools for sterilization.

FIG. 7 illustrates a block diagram of a system for performing image analytics using machine learning according to certain aspects of the present disclosure.

FIG. 8 illustrates a block diagram of a system for displaying augmented reality information according to certain aspects of the present disclosure.

### DETAILED DESCRIPTION

In the following description, the present disclosure is set forth in the context of various alternative embodiments and implementations involving methods and systems to track surgical tools, e.g., medical and surgical devices, equipment, and consumables, through an entire use cycle. As used herein, a use cycle includes at least the steps of cleaning (i.e., decontamination), assembling, sterilizing, and transporting the tools to a use location, such as an operating room or other location of an aseptic procedure, or for storage. A use cycle may further include the steps of collecting the tools after an aseptic procedure and transporting them to a location for decontamination.

The present disclosure is further set forth in the context of methods and systems to optimize assembly of surgical tool set for use at various locations in a perioperative environment based on tracking of use rate or non-use of individual surgical tools during an aseptic procedure.

While the following description discloses numerous exemplary embodiments, the scope of the present patent application is not limited to the disclosed embodiments, but also encompasses combinations of the disclosed embodiments, as well as modifications to the disclosed embodiments.

Various aspects of the systems and methods may be illustrated by describing components that are coupled, attached, and/or joined together. As used herein, the terms “coupled”, “attached”, and/or “joined” are interchangeably used to indicate either a direct connection between two hardware components, or two software modules, or, where appropriate, an indirect connection to one another through intervening or intermediate components or modules. In contrast, when a component is referred to as being “directly coupled”, “directly attached”, and/or “directly joined” to another component or module, there are no intervening elements in said examples.

Various aspects or embodiments of the systems and methods may be described and illustrated with reference to one or more exemplary implementations. As used herein, the term “exemplary” means “serving as an example, instance, or illustration,” and should not necessarily be construed as preferred or advantageous over other variations of the devices, systems, or methods disclosed herein. “Optional” or “optionally” means that the subsequently described event or circumstance may or may not occur, and that the description includes instances where the event occurs and instances where it does not. In addition, the word “comprising” as used herein means “including, but not limited to”.

Unless otherwise specified, expressions “approximately”, “substantially”, and “in the order of” mean to within 20%, such as to within 10%, or even to within 5%.

It must also be noted that as used herein and in the appended claims, the singular forms “a”, “an”, and “the” include the plural reference unless the context clearly dictates otherwise. Unless defined otherwise, all technical and scientific terms used herein have the same meanings as commonly understood by one of ordinary skill in the art.

As used herein, the terms user, staff, and personnel may all be understood to refer to any person working in the perioperative environment, any person providing or assisting an aseptic procedure, or any person using, transporting, cleaning, storing, or logging information related to surgical tools.

The flowchart and block diagrams in the figures illustrate the architecture, functionality, and/or operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved.

Words such as “then,” “next,” etc. are not intended to limit the order of the steps; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

Features or functionality described with respect to certain example embodiments may be combined and sub-combined in and/or with various other example embodiments. Also, different aspects and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, several steps may be required before, after, and/or concurrently with example embodiments, as disclosed herein. Note that any and/or all methods and/or processes, at least as disclosed herein, can be at least partially performed via at least one entity or actor in any manner.

Provided herein are systems, methods, and non-transitory computer-readable media for tracking the path of sterilizable tools through the perioperative environment, and for optimizing the provision or assembly of surgical tools for use in specific aseptic procedures. The tracking may be at specific locations in the perioperative environment, such as before and after an aseptic procedure, e.g., in the operating room, or at more than one location in the perioperative environment, such as in the operating room, the decontamination area, and the assembly area. The tracking may be in a specific region of the area, such as on entry and/or exit (e.g., at the door), or entry and/or exit from a specific region in the area (e.g., position within the OR, entry/exit from the washers, entry/exit from an assembly area, entry/exit from the sterilization equipment, etc.). The tracking may be in real-time in any of these areas. The tracking may be in real time throughout substantially the entire path of a use cycle in the perioperative environment.

As used herein, the perioperative environment includes at least the sterile processing depat intent (SPD), central supply department (CSD), and operating room or other location of an aseptic procedure (collectively referred to herein as OR for simplicity).

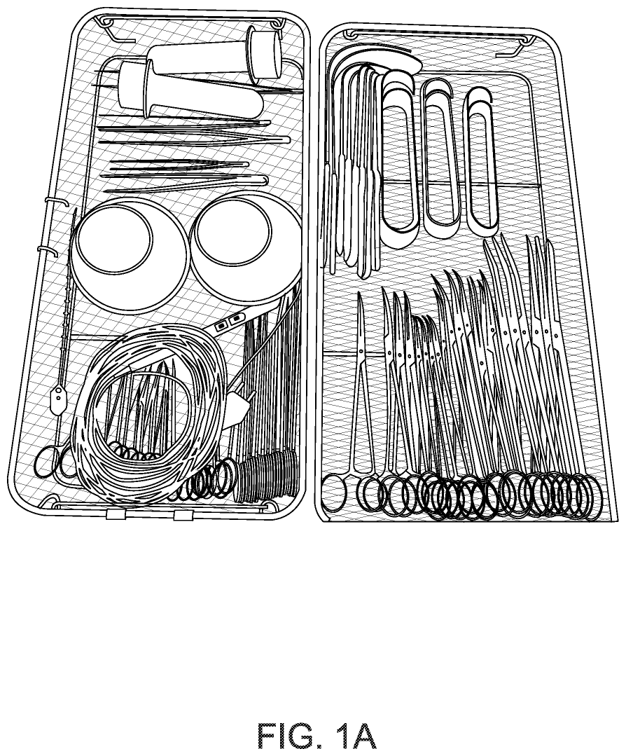

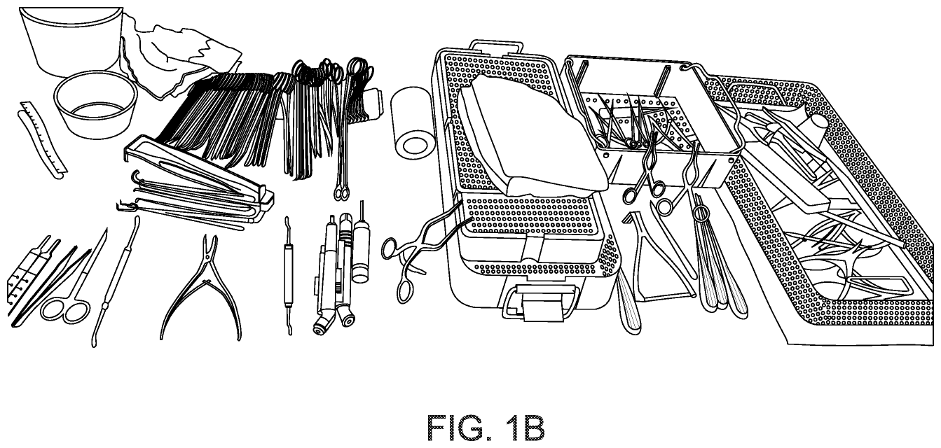

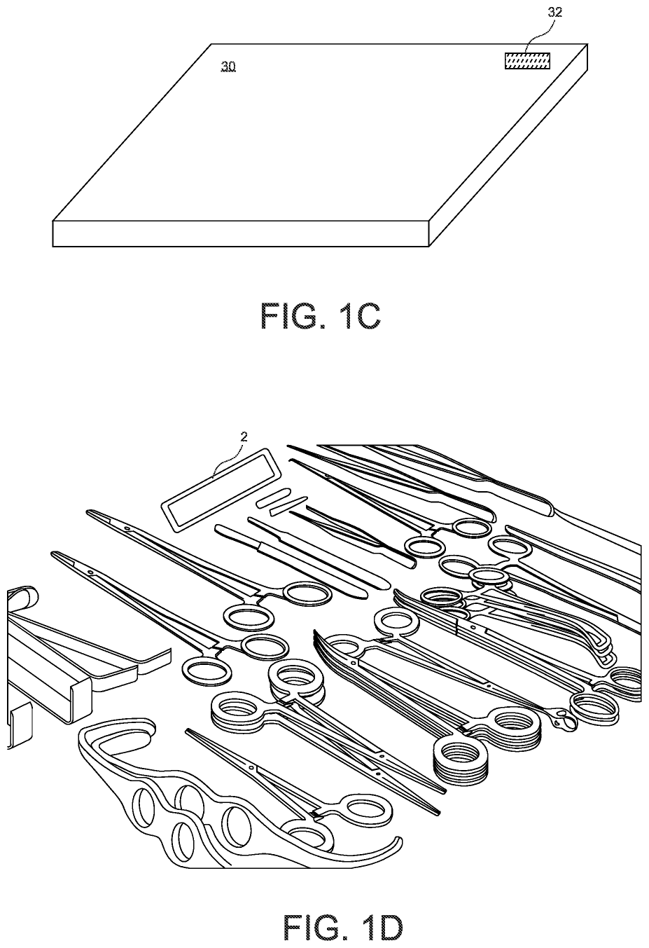

Tools such as surgical tools and devices are provided within the OR in a specific number, arrangement, and range depending on the procedure and the surgeon performing the procedure. An exemplary arrangement of surgical tools within an OR is shown in FIG. 1B. Each tool is included on a specific table at a preferred position so that the tool is available and quickly retrievable during surgery. After use of the tool, it may be returned to that position or placed on another tray, but in both cases, all tools must be accounted for and processed after the procedure is complete. Thus, the surgical staff must count each tool to be sure that all are present before the surgeon closes the incision and/or before the tools are scanned out of the OR, such as shown in step 12 of FIG. 4. Accordingly, the speed at which the tools can be counted directly impacts not only the patient but the turn-around time for the OR.

The present invention provides methods and systems that greatly enhance the accuracy and speed at which at least this step may be accomplished. Specifically, the methods and systems disclosed herein use computer vision to locate, identify, and track tools such as surgical tools and devices through the perioperative environment. Thus, at the end of a surgical or aseptic procedure such as in an OR, computer vision may be used to rapidly identify the tools in the OR, and to indicate specific tools that are missing, if any. The method may further account for inclusion of tools in a plurality of surgical tools, e.g., a tray configured for a specific surgeon or aseptic procedure, that are extra. For example, during assembly of a tray comprising a plurality of surgical tools intended for a specific procedure or surgeon, should tools not on an intended list be noted, they may be removed from the tray before the tools are sterilized and stored or distributed within the perioperative environment.

While decontamination and sterilization of surgical tools is necessary, it reduces the lifespan of the surgical tool. When a plurality of surgical tools is provided for an aseptic procedure on a tray, for example, those tools not used during the procedure must be processed in the same manner as those that were used, i.e., they must all be decontaminated and sterilized at the end of the aseptic procedure. Thus, the methods and systems disclosed herein are further configured to track tool use and may provide for omission from the surgical tray those tools not used or rarely used during the procedure. These methods may reduce wear on the omitted tools and extend their lifespan. The rarely or never used tools may be provided in individual sterilized packaging as a precaution should they become needed in the aseptic procedure.

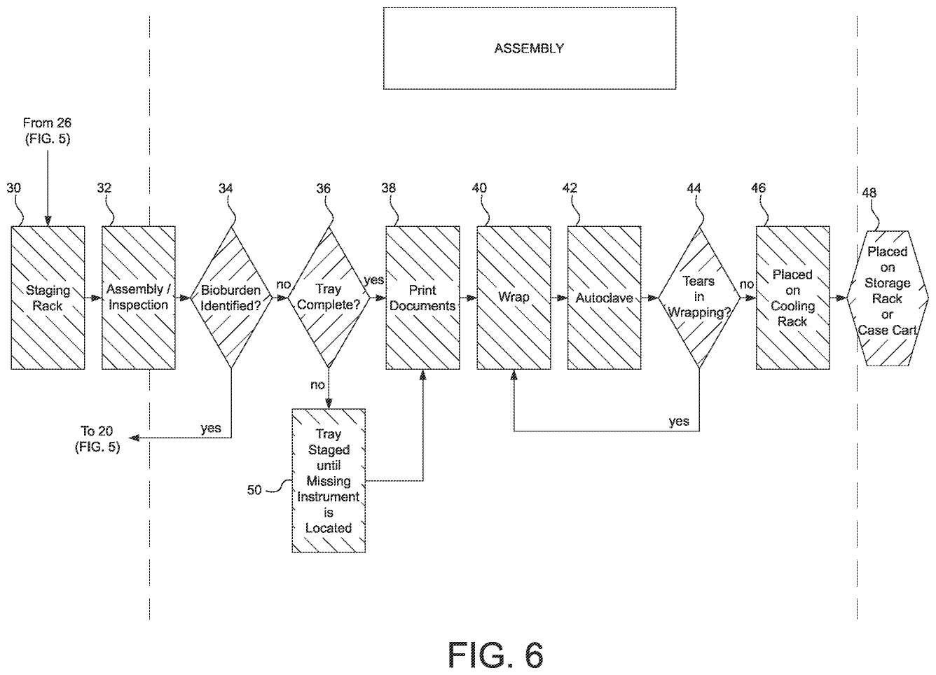

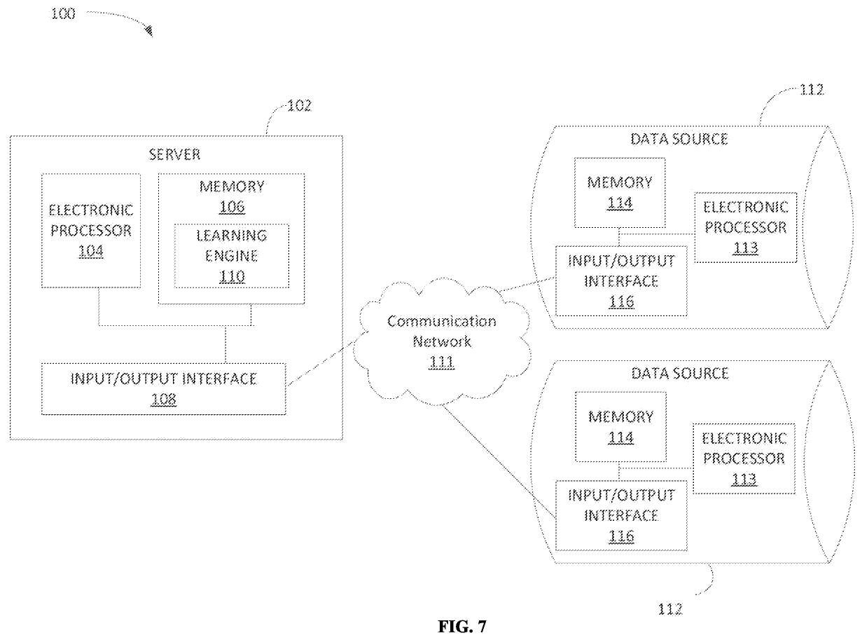

With reference to FIGS. 4-6, a standard use cycle in the perioperative environment is detailed. At the end of a surgical procedure, such as in an OR (10), all tools are scanned out of the OR (12), after which that may be transported and scanned into the soiled utility room (14). From there, the tools will again be transported (16) and scanned (18) into a decontamination area (20; FIG. 5).

Once in the decontamination area (20), the tools are rinsed (22), soaked (24), scrubbed (26), and placed into racks or trays for machine washing (28). The clean tools may then be positioned on staging racks (32) in an assembly area where they are inspected for accurate content (34) and are passed into the assembly area (30) where the contents of the racks or trays are reviewed and checked for cleanliness (36). If further cleaning is required, all or some of the tools may be sent back for additional cleaning (such as in steps 22-28). Alternatively, if the assembled tools are considered ready for sterilization, such as shown in FIG. 1A, they may be further inspected to be sure that the tray is complete (38). If not found to be complete, i.e., comprising all expected tools, the tray may be staged (40) until such time as all tools are available.

Once the tray is determined to contain the expected complement of tools, it may be wrapped (44) and sterilized (46), such as by autoclave. Documents indicating the tools on the tray and the next use of the tools, i.e., the surgeon, procedure, and scheduled OR date and time, may be printed (42) prior to wrapping and sterilization. Once sterilized, the wrapping may be inspected for any defects or tears that may negatively impact the sterility of the wrapped tools (48), and if any defects are found, the tray may be re-wrapped (44), sterilized (46) and re-inspected for defects (48). If, however, the wrapping has remained intact, the tray may be placed on a cooling rack (50) prior to storage or transport to an OR for use (52).

In a facility using prior art methods and systems, each tool has remained untracked, such as during transport and washing, or has been hand counted and tracked on paper or by manual entry into a computer system or by verbal feedback at only a few steps in the process, such as upon exit from the OR, entry to the decontamination facility, and/or during assembly of the trays. The risk for tool loss is relatively great and becomes exponentially larger for each step that remains untracked. Moreover, the manual nature of counting and logging each tool at multiple steps is not only time consuming but labor intensive.

The methods and systems of the present disclosure remove most if not all of these points of potential loss and offer great time savings by providing automatic methods and systems for tool tracking and optimization of tool assemblies. That is, the systems and methods utilize computer vision for automated recognition and recording of the tools at one or more points within the perioperative environment, e.g., as they transit through the perioperative environment. The tracking may be in real time at some or all of the locations and may even be continuous throughout a substantial portion of the transit throughout the perioperative environment.

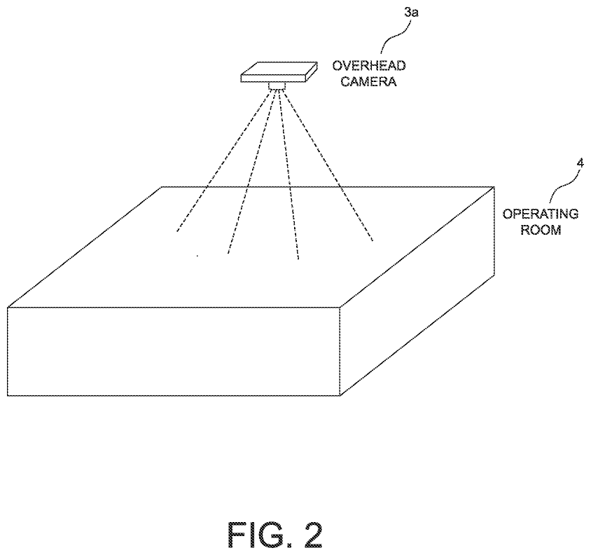

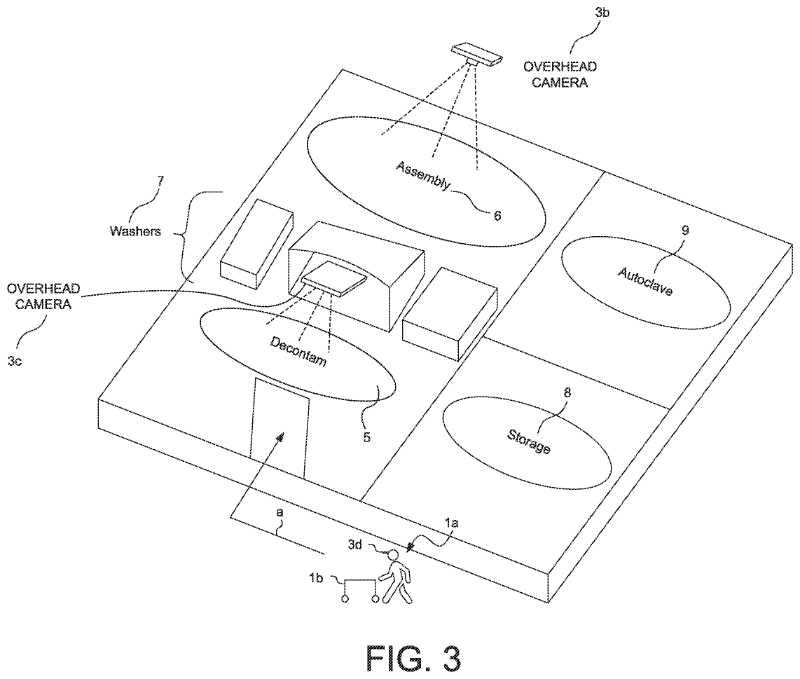

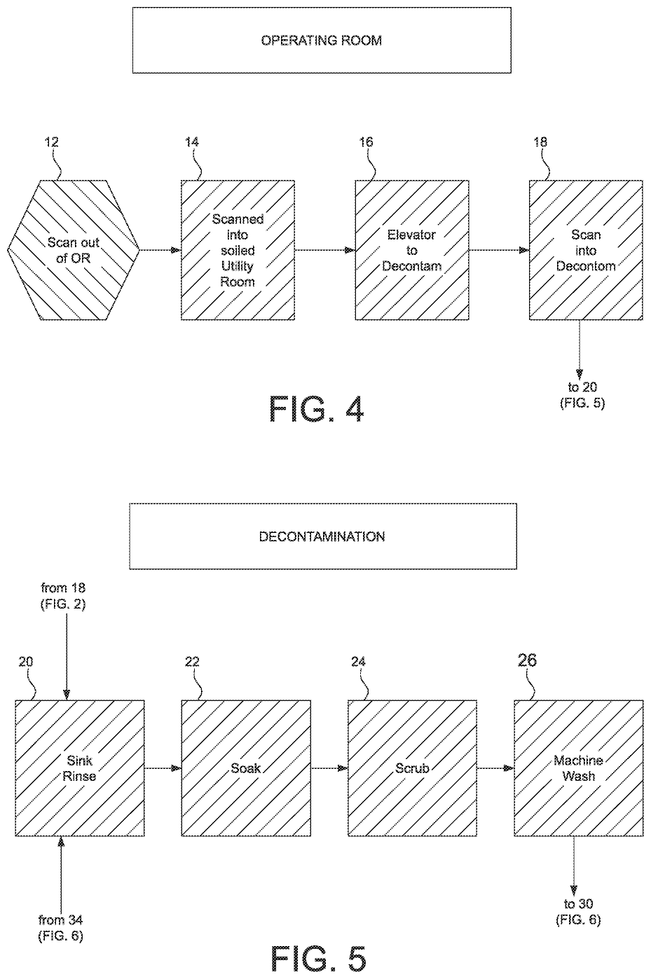

With reference to FIGS. 2 and 3, the computer vision may be implemented through use of one or more cameras, such as at least one camera 3a in an OR 4, one camera 3b in a decontamination area 5, and one camera 3c in an assembly area 6. Additional cameras may also be provided throughout other regions of the perioperative environment, i.e., hallways, elevators, storage areas, etc. typically the cameras provided in these areas are mounted above the user transporting, processing, or using the surgical instruments, such as on a ceiling or high up on a wall. According to certain aspects, the camera may be room mounted (ceiling, on a poll extending from the ceiling, high up on a wall).

The cameras could alternatively or additionally include an augmented reality system or device or a body camera (human worn camera, 3d), such as worn by one or more members of personnel 1a in the perioperative environment. Cameras 3d worn by personnel in the perioperative environment may be able to view the surgical tools on a tray or cart 1b throughout additional transit steps (e.g., path ‘a’ shown in FIG. 3) and/or locations (i.e., hallways, elevators, loading and unloading from the washers 7 and/or autoclave 9, placement into storage 8, etc.).

Thus, using the presently disclosed systems and methods, the tool tracking requires no manual counting or logging of tool numbers and identities. Moreover, when setting up specific racks or trays in the assembly area of the perioperative environment, the presently disclosed system and methods may automatically guide personnel to position tools in the correct number and arrangements, such as required by a specific surgeon, procedure, and/or scheduled OR date and time during which the tray will be used.

While certain embodiments of the methods and systems are described herein as executing a task, e.g., guiding personnel to perform a function, every method and system may also be provided for the purpose of supporting personnel in their functions. That is, the systems and methods may be viewed as assistive to the functions normally carried out in a perioperative environment.

The computer vision may be linked to a platform that utilizes machine learning to optimize the process. Machine learning generally refers to the ability of a computer program to learn without being explicitly programmed. According to some aspects, a computer program (e.g., a learning engine) is configured to construct a model (e.g., one or more algorithms) based on example inputs, such as through supervised learning. Supervised learning involves presenting a computer program with example inputs and their desired (e.g., actual) outputs. The computer program is configured to learn a general rule (e.g., a model) that maps the inputs to the outputs.

The methods and programs of the present disclosure may be configured to perform machine learning using various types of methods and mechanisms. For example, the methods and programs may perform machine learning using decision tree learning, association rule learning, artificial neural networks, inductive logic programming, support vector machines, clustering, Bayesian networks, reinforcement learning, representation learning, similarity and metric learning, sparse dictionary learning, and genetic algorithms. Using all of these approaches, a computer program may ingest, parse, and understand data and progressively refine models for data analytics.

The present disclosure also relates to methods and systems for providing training information to a learning engine to properly train the learning engine to automatically and continuously identify and track specific tools, update surgeon preference cards accordingly, and ultimately provide predictive algorithms for specific toolkits and/or surgical trays, i.e., collections of tools tailored for specific surgeons and surgical procedures. That is, the system may also track usage of tools in an OR, i.e., which tools are used and which remain unused, and may provide feedback regarding optimization of the surgical tools provided in a tray for each surgeon and/or procedure.

Accordingly, methods and systems of the present invention may be configured to capture images substantially in real time, e.g., video, and may include analysis of the images to track removal and/or movement of individual surgical tools in the plurality of surgical tools (e.g., individual tools on a tray of tools). For example, the images or video may be processed and analyzed to track hand motions, or may be processed and analyzed to compare location, identity, and number of surgical tools as a function of time. This comparison may show which tools are never or only rarely used. The never or rarely used tools may be omitted from future surgical tool sets, and/or may be provided in separate sterilized packaging so that they are available in the rare instance that they are required. This allows the surgical sets to be streamlined, and further provides reduced wear on those generally unused and/or rarely used surgical tools (i.e., wear from decontamination and sterilization).

As mentioned hereinabove, the methods and systems provided herein may expedite counting and removal of surgical tools after an aseptic procedure. The presently disclosed methods and systems may also expedite movement of the surgical tools through the perioperative environment, such as by reducing the time required to track tools after decontamination (washing) and during assembly. The method may suggest tool lists and/or positioning of tools on trays to assist the personnel.

The methods and systems provided herein may be used to detect lapses in sterile handling of the surgical tools, such as detecting holes or openings in a wrapper surrounding a sterilized tray, mishandling of sterilized items on a tray in the perioperative environment, and the like. The methods and systems provided herein may be also be used to detect possible debris remaining on a tool after decontamination (washing), before assembly, before sterilization, after sterilization, and before use in an aseptic procedure. Various cameras positioned in any of these areas, or anywhere in the perioperative environment, may be configured to detect such contamination (i.e., infrared and/or ultraviolet cameras).

Moreover, data related to recorded hand motions over the plurality of surgical tools, such as over a tray during an aseptic procedure to remove and replace tools thereon, may also be used to train new personnel as they learn how a specific aseptic procedure is completed, or how a specific surgeon prefers tools to be handed off. Moreover, the data related to recorded hand motions over the plurality of surgical tools, may be acquired in any region of the perioperative environment, such as during assembly, decontamination, or in the IR. Accordingly, methods and systems of the present invention may be configured to provide training information useful to educate personnel in the perioperative environment. The training information may relate to use and/or placement of individual surgical tools in the OR, best practices for decontamination, proper assembly of trays for specific procedures and/or specific surgeons, and the like.

For example, as shown in FIG. 7, a system 100 for performing machine learning according to certain aspects of the present disclosure may include a server 102 having a plurality of electrical and electronic components that provide power, operational control, and protection of the components within the server 102. The server 102 may include an electronic processor 104 (e.g., a microprocessor, application-specific integrated circuit (ASIC), or another suitable electronic device), a memory 106 (e.g., a non-transitory, computer-readable storage medium), and an input/output interface 108. The electronic processor 104, the memory 106, and the input/output interface 108 may communicate over one or more connections or buses. The server 102 illustrated in FIG. 7 represents one example of a server and embodiments described herein may include a server with additional, fewer, or different components. Also, according to certain aspects, the server 102 performs functionality in addition to the functionality described herein. Similarly, the functionality performed by the server 102 (i.e., through execution of instructions by the electronic processor 104) may be distributed among multiple servers. Accordingly, functionality described herein as being performed by the electronic processor 104 may be performed by one or more electronic processors included in the server 102, external to the server 102, or a combination thereof.

The memory 106 may include read-only memory (“ROM”), random access memory (“RAM”) (e.g., dynamic RAM (“DRAM”), synchronous DRAM (“SDRAM”), and the like), electrically erasable programmable read-only memory (“EEPROM”), flash memory, a hard disk, a secure digital (“SD”) card, other suitable memory devices, or a combination thereof. The electronic processor 104 executes computer-readable instructions (“software”) stored in the memory 106. The software may include firmware, one or more applications, program data, filters, rules, one or more program modules, and other executable instructions. The software may include instructions and associated data for performing the methods described herein. For example, as illustrated in FIG. 7, the memory 106 may store a learning engine 110 (i.e., software) for performing image analytics as described herein (e.g., processing training information to develop models). However, according to certain aspects, the functionality described herein as being performed by the learning engine 110 may be performed through one or more software modules stored in the memory 106 or external memory.

The input/output interface 108 allows the server 102 to communicate with devices external to the server 102. For example, as illustrated in FIG. 7, the server 102 may communicate with one or more data sources 112 through the input/output interface 108. In particular, the input/output interface 108 may include a port for receiving a wired connection to an external device (e.g., a universal serial bus (“USB”) cable and the like), a transceiver for establishing a wireless connection to an external device (e.g., over one or more communication networks 111, such as the Internet, a local area network (“LAN”), a wide area network (“WAN”), and the like), or a combination thereof.

According to certain aspects, the server 102 also receives input from one or more peripheral devices, such as a keyboard, a pointing device (e.g., a mouse), buttons on a touch screen, a scroll ball, mechanical buttons, computer vision devices and/or cameras (e.g., cameras 3a-3c), and the like through the input/output interface 108. Similarly, according to certain aspects, the server 102 provides output to one or more peripheral devices, such as a display device (e.g., a liquid crystal display (“LCD”), a touch screen, and the like), an augmented reality device (e.g., headset, visor, glasses, e.g., 3d), a printer, a speaker, and the like through the input/output interface 108. According to certain aspects, output may be provided within a graphical user interface (“GUI”) (e.g., generated by the electronic processor 104 executing instructions and data stored in the memory 106 and presented on the augmented reality device) that enables a user to interact with the server 102 (e.g., through perceived gestures of a user wearing the device).

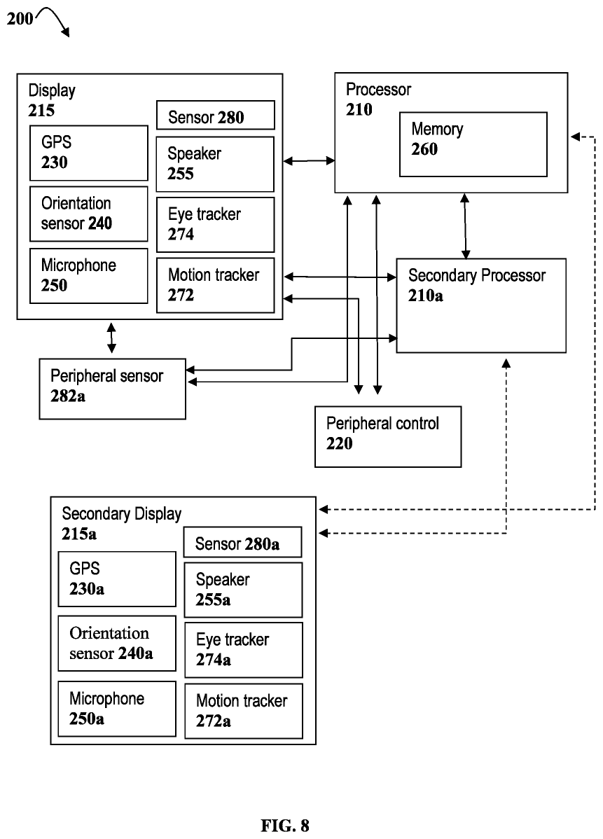

According to certain aspects, a user may interact with the server 102 through one or more of a keyboard, a mouse, or an intermediary device, such as a personal computing device laptop, desktop, tablet, smart phone, smart watch or other wearable, smart television, augmented reality or “AR” device, and the like). For example, a user may configure functionality performed by the server 102 as described herein by providing data to an intermediary device that communicates with the server 102. In particular, a user may use voice commands or gesture commands perceived by the AR device (200, see FIG. 8) that are communicated to the server 102. As another example, a browser application executed by an intermediary device may access a web page that receives input from and provides output to the user for configuring the functionality performed by the server 102.

As illustrated in FIG. 7, the system 100 includes one or more data sources 112. Exemplary data sources include databases storing reference information, such as image data and/or data related to pattern recognition of surgical tools (size, shape, etc.), data relating a code such as found on a 2D tag on the tray (tag 32 on tray 30; of FIG. 1C) or surgical tool to specific information, and the like (i.e., any reference data that may assist the methods and systems in recognition of surgical tools).

Further exemplary data sources include historic and/or current tracking data for use of specific tools sets (i.e., sets of surgical tools provided per tray or per aseptic procedure) in an aseptic procedure or transit of specific tools sets throughout the perioperative environment. The data may be categorized by date, time, procedure, specific location (e.g., OR, decontamination area, assembly area), surgeon or other personnel, surgical tool type or specific surgical tool identity or brand, and the like. In this way, the systems and methods disclosed herein may not only assist in tracking surgical tools, such as during any step in the perioperative environment, but may also assist in (i) optimizing tools included in specific tools sets (e.g., based on an aseptic procedure, a surgeon, etc.), (ii) tracking loss points within the perioperative environment (e.g., during washing), (iii) informing staffing needs, (iv) informing surgical tool use and longevity (e.g., based on type, brand, procedure), (v) informing surgical tool needs, and the like.

The data collected using the methods and systems disclosed herein may be filtered to protect patient privacy. For example, while the data collected for tracking and/or recognition of surgical tools is described as received from a camera, such as a camera providing video, the raw images or video may not be stored. That is, the data may be logged in a data source 112 in the form of identities of surgical tools, locations of the surgical tools, and times of noted identities and/or locations.

According to certain aspects, the data may include video or still images of only the tray or cart comprising the surgical tools, i.e., exclude any image not comprising the tray or cart. This may be accomplished via markers positioned on the tray or cart that define a boundary for image recording or detection. Such may enhance privacy and may also enhance efficiency of the processing step as large regions of images or video may not need to be analyzed.

Each data source 112 may include a plurality of electrical and electronic components that provide power, operational control, and protection of the components within the data source 112. According to certain aspects, each data source 112 represents a server, a database, a personal computing device, an AR device, or a combination thereof. For example, as illustrated in FIG. 7, each data source 112 may include an electronic processor 113 (e.g., a microprocessor, ASIC, or other suitable electronic device), a memory 114 (e.g., a non-transitory, computer-readable storage medium), and an input/output interface 116. The data sources 112 illustrated in FIG. 7 represent one example of data sources and embodiments described herein may include a data source with additional, fewer, or different components than the data sources 112 illustrated in FIG. 7. Also, according to certain aspects, the server 102 communicates with more or fewer data sources 112 than illustrated in FIG. 7.

The input/output interface 116 allows the data source 112 to communicate with external devices, such as the server 102. For example, as illustrated in FIG. 7, the input/output interface 116 may include a transceiver for establishing a wireless connection to the server 102 or other devices through the communication network 111 described above. Alternatively, or in addition, the input/output interface 116 may include a port for receiving a wired connection to the server 102 or other devices. Furthermore, according to certain aspects, the data sources 112 also communicate with one or more peripheral devices through the input/output interface 116 for receiving input from a user, providing output to a user, or a combination thereof. According to certain aspects, one or more of the data sources 112 may communicate with the server 102 through one or more intermediary devices. Also, according to certain aspects, one or more of the data sources 112 may be included in the server 102.

As indicated herein, methods of the present disclosure are configured to track a plurality of surgical tools through all or a portion of a use cycle. The methods generally comprise use of computer vision, such as supplied by one or more cameras or an AR device, to capture one or more images of a tool, such as one or more surgical tools, or even a plurality of surgical tools. These images may be processed, such as by a processor of the AR device 113 or by a processor of a server 102, to determine the identities of each surgical tool in the plurality of surgical tools, and such identities may be recorded in short-term memory of either the AR device or the server, or in a database on the server.

Thus, according to certain aspects, the methods and systems of the present disclosure may comprise at least one camera and/or at least one AR device wearable by a user responsible for transit or handling of the surgical tools within the perioperative environment. When the system includes one or more cameras, or includes data collected from one or more cameras, they may provide still images collected at specific time intervals (e.g., every 1 second) or they may provide continuous real time video stream. When the system includes one or more AR devices, the AR devices may provide computer vision to intermittently or continuously capture images of the surgical tools. In many cases, more than one user may interact with the surgical tools during their transit through the perioperative environment, and thus, each of those users who handle the tools may also be provided with an AR device configured to capture images of the surgical tools.

According to aspects of the present disclosure, the methods may further comprise comparing the recorded identities of each surgical tool to an expected list of identities of the plurality of surgical tools. The expected list of identities may be determined based on a user input value or a fiducial marker found in the one or more images captured by the camera. For example, the user may manually input the expected list of surgical tools or may provide a code related to a stored list of expected tools, such as a list name. Alternatively, or in addition, the tools may be supplied on a tray that includes a visual maker that may link to a stored list of expected tools, such as a 2D code. Exemplary 2D codes include at least bar codes, ArUco codes, QR codes, and the like.

The present methods may use pattern recognition of a shape and size of each surgical tool and/or a tag attached to each surgical tool to determine an identity of the surgical tool. Alternatively, or in addition, the present methods may use a machine learning algorithm to train the system to recognize individual surgical tools, such as described hereinabove.

Accuracy of tracking may be generally improved with continuous or extended tracking throughout the entire use cycle of each surgical tool. That is, computer vision may be used to identify and track each tool not only at specific steps in the use, cleaning, assembly, and sterilization process, but also during some or all of the transport steps (i.e., on exit from the OR, entry to the decontamination facility and sterilization facility, on transport to storage or to a new OR, etc.). Accordingly, the steps of capturing and processing image data, and recording identity data may be completed upon each transfer of the plurality of surgical tools through the perioperative environment. According to other aspects, these steps may be completed substantially continuously as the plurality of surgical tools are transported throughout a perioperative environment.

Image capture may use computer vision, such as cameras that are positioned about the OR or perioperative environment. The camera(s) may capture visual images, infrared images, thermal images, lidar images, and/or any other known real-time images that may be used to extract shape and size data for use in processing to determine a surgical tool identity. The camera(s) may transmit captured images to a processor, which may compare the captured images to reference images, and may record an identity of one or more surgical instruments shown in the captured image (e.g., such as by an AR device or camera worn on personnel in the perioperative environment).

As the surgical tools are tracked throughout the perioperative environment, the methods disclosed herein may determine if one or more surgical tools are missing. This information, which may include at least a number of missing tools and/or an identity of the one or more missing tools, may be communicated via an output device of the server 102, such as a graphical user interface (screen or AR device). For example, according to certain aspects, the methods of the present disclosure may further include creating augmented reality information comprising an identity of each of the one or more missing surgical tools, and outputting that information in a field of view of a user, such as on a display unit of the AR device. An exemplary display includes at least a head mounted transmissive display.

According to certain aspects, the expected number and identity of the surgical tools may be related to a specific tray setup, such as defined by a surgeon's preference card, a type of procedure, a specific OR, and/or based on certain patient specific characteristics. Accordingly, the methods may include capturing one or more images of the plurality of surgical tools as they are assembled in a tray. The AR device may be configured to output the information comprising an identity of each of the one or more missing surgical tools in response to a user placing the assembled tray having the tool missing in a field of view of the user or of a camera, whereupon an identity and/or correct location of the one or more missing surgical tools may be perceivable in the field of view of the user or on a display device. For example, the identity and/or correct location of the one or more missing surgical tools may be overlaid on the assembled tray, such as the box 2 shown in FIG. 1D.

As indicated above, the systems and methods of the present disclosure may use machine learning algorithms for identification of specific surgical tools from the captured images. The determination that a tool is missing may be based on specific lists of expected tools, such as a list of tools that entered an OR, wherein the same list of tools must be identified on exit from the OR. Additionally, ale expected lists of toots may be based on surgeon preference lists or “cards”. While these lists have generally remained static in prior art uses, the systems and methods of the present disclosure may use machine learning algorithms to update such lists. For example, the system and methods may identify not only the specific surgical tools in ale OR or perioperative environment but may be configured to identify the surgical tools that have been used and those that have remained unused. As such, a surgeon's preference card may be tailored based on real-time use data and statistics, and further tailored based on specific procedures, OR's, and/or patient characteristics.

Establishing an augmented reality (AR) display within a real space refers to using computer generated virtual objects projected into the space, where the virtual objects behave as if they are physically in the space, and where one or more users may be able to see each other (i.e., actual or virtual representations) and the virtual objects, and interact with the virtual objects and each other. One of the key aspects of the methods and systems of the present disclosure is the use of computer vision to capture and analyze images of the surgical tools as they transit within a perioperative environment. The cameras of the computer vision may be supplied by an AR device, such as indicated hereinabove.

As shown in FIG. 8, an AR device 200 of the present disclosure may include a processor 210, a memory 260, and a display 215. According to certain aspects, the AR device 200 may additionally include one or more secondary processors 210a, one or more secondary displays 215a, a peripheral control 220, a global positioning system (GPS) 230, an orientation sensor 240, a microphone 250, and/or a speaker 255. Alternatively, these may be included as a separate component. As shown in FIG. 8, each of the GPS 230, orientation sensor 240, and microphone 250 may be a part of the display 215, wherein the display 215 may be in electronic communication with either or both processors (210, 210a). Moreover, when included, the peripheral control 220 may be in electronic communication with the display 215 and/or the processor(s) (210, 210a).

The peripheral control 220 may refer to a remote control, such as a hand-held unit that may provide or allow manual selection (e.g., via buttons or IR) of options. According to certain aspects, the peripheral control 220 includes a joystick. The orientation sensor 240 determines the gyroscopic orientation of the user and/or display unit 215 and may enable the system to determine the angle the user is looking. The GPS 230 may be included to further aid in detecting movement of the user and/or display unit 215. The orientation sensor 240 and/or GPS 230 may be included on a plurality of suitable display devices (AR devices).

The microphone 250 may enable the user to provide auditory cues when applicable to tasks performed, such as to record a current location of the surgical tools or devices. The speaker 255 may enable the user to receive auditory cues when applicable to tasks performed, such as a changed destination of the surgical tools, a change in the content of the surgical tools on a transported tray, etc.

Additional elements of the system of the present disclosure may include a motion tracker 272 and eye tracker 274, which may be provided to improve the image capture and information projection (i.e., identity and position on a tray of the missing surgical tool). Moreover, one or more additional sensors (280, 280a) may be included as part of the AR device or separate from the AR device. These additional sensors may be in electronic communication with the processor 210 (or server 102) and may provide additional information that may assist ire tracking movement of the user or real objects in the perioperative environment or may assist in defining the OR environment (e.g., camera that may view the actual OR environment in an augmented reality simulation).

According to certain aspects, the AR device may include one or more screens, such as a single screen or two screens (e.g., one per eye of a user). The screens may allow light to pass through the screens such that aspects of the real environment are visible while displaying a virtual object. The virtual object may be made visible to the user by projecting light. The virtual object may appear to have a degree of transparency or may be opaque (i.e., blocking aspects of the real environment). According to certain aspects, the user of the system may interact with the virtual object, such as by moving the virtual object from a first position to a second position.

Detection of actions and interactions may involve the use of wearable or freestanding sensors, such as cameras, infrared (IR) beacons, wireless beacons, and inertial measurement units attached to the AR device. These sensor(s) may be communicatively coupled to a computer. The sensor(s) may be configured to provide data (e.g., image data, sensed data, six degrees of freedom data, etc.) to the computer (server 102, processor 210). Furthermore, the sensor(s) may be configured to receive data (e.g., configuration data, setup data, commands, register settings, etc.) from the computer.

Aspects

The following aspects are provided by the present disclosure and form at least a portion of the presently disclosed invention:

A tracking method for a plurality of surgical tools through a use cycle, the method comprising: capturing one or more images of the plurality of surgical tools while in a perioperative environment; processing the one or more images to determine identities of each surgical tool in the plurality of surgical tools; recording the determined identities of each surgical tool to form an initial list of identities; and comparing the initial list of identities of each surgical tool to an expected list of identities.

The tracking method above wherein the expected list of identities is determined based on a user input value or a fiducial marker found in the one or more images captured by the camera. The method wherein the fiducial marker is a 2D code. The method wherein the data associated with the fiducial marker comprises information related to a date/time a tray was assembled, information related to aspects of the sterilization process (time/temp/method), information related to personnel who assembled and/or sterilized the tray, or any combination thereof.

Any of the tracking methods above wherein the step of processing the one or more images to determine identities of each surgical tool in the plurality of surgical tools comprises: pattern recognition of a shape and size of each surgical tool, a tag attached to each surgical tool, or a combination of both. The method wherein the tag is a 2D code.

Any of the tracking methods above wherein the step of processing the one or more images to determine identities of each surgical tool in the plurality of surgical tools comprises: comparing the one or more images of the plurality of surgical tools to stored images of a plurality of reference surgical tools; and generating a comparison score for each of the plurality of surgical tools based on the comparing step, wherein the identity of each of the plurality of surgical tools is assigned as an identity of the reference surgical tool having the highest comparison score.

Any of the tracking methods above wherein the step of capturing one or more images of the plurality of surgical tools comprises receiving image data from a camera. The method wherein the camera comprises a 3D camera, a color camera, an IR camera, a UV camera, lidar, or a combination thereof.

Any of the tracking methods above wherein the camera is a body mounted camera, a room mounted camera, or a combination thereof.

Any of the tracking methods above wherein the plurality of surgical tools are arranged on a tray, and the step of capturing the one or more images further comprises capturing an image of a reference tag on the tray, wherein the reference tag provides: a reference grid for obtaining dimensions of the plurality of surgical tools and/or a 2D code that identifies the expected list of identities of surgical tools on the tray.

Any of the tracking methods above wherein the step of processing the one or more images to determine the identity of each surgical tool in the plurality of surgical tools comprises: recognizing a reference tag positioned on each of the surgical tools and accessing a reference list database comprising a surgical tool identity related to the reference tag.

Any of the tracking methods above wherein the steps of capturing, processing, and recording are completed at more than one location in the perioperative environment, wherein the perioperative environment includes at least an operating room, a decontamination area, and an assembly area. The method wherein the steps of capturing, processing, and recording are completed at each of the operating room, the decontamination area, and the assembly area.

Any of the tracking methods above wherein the steps of capturing, processing, and recording are completed substantially continuously as the plurality of surgical tools are transported throughout the perioperative environment.

Any of the tracking methods above further comprising, based on the comparing step, determining one or more surgical tools missing from the plurality of surgical tools, one or more surgical tools added to the plurality of surgical tools, or a combination of both.

Any of the tracking methods above further comprising, based on the comparing step, determining one or more surgical tools missing from the plurality of surgical tools, one or more surgical tools added to the plurality of surgical tools, or a combination of both.

Any of the tracking methods above comprising creating an augmented reality information comprising an identity of each of the one or more surgical tools missing from the plurality of surgical too, added to the plurality of surgical too, or a combination of both; and outputting the augmented reality information in a field of view of a user (e.g., on a computer screen or on an AR device worn by the user).

Any of the tracking methods above wherein the step of capturing an image of the plurality of surgical tools comprises capturing an image of an assembled tray comprising the plurality of surgical tools.

Any of the tracking methods above further comprising, creating an augmented reality information that relates to one or more missing surgical tools from the assembled tray by evaluating, via a machine learning algorithm, the image captured by the camera of the assembled tray; and outputting the augmented reality information such that, in response to a user placing the assembled tray in a field of view of the user, an identity and/or correct location of the one or more missing surgical tools is perceivable in the field of view of the user (e.g., on a computer screen or on an AR device worn by the user).

Any of the tracking methods above wherein the identity and/or correct location of the one or more missing surgical tools is provided as an overlaid image on the assembled tray in the field of view of the user.

Any of the tracking methods above wherein the identity and/or correct location of the one or more missing surgical tools is based on a surgeon preference list, a procedure type, or both. The method wherein the surgeon preference list includes a list of surgical tools selected by a surgeon, or a list of surgical tools determined by the machine learning algorithm.

Any of the tracking methods above wherein the list of surgical tools determined by the machine learning algorithm is identified by a use rate of individual surgical tools in the plurality of surgical tools during a procedure in an aseptic environment.

Any of the tracking methods above wherein the step of capturing one or more images of the plurality of surgical tools is performed in an operating room prior to initiation of an aseptic procedure, and the method further comprises, during the aseptic procedure, repeating the capturing and processing steps substantially in real-time, and recording an identity of each surgical tool removed from the plurality of surgical tools to form a list of surgical tools used during the aseptic procedure.

Any of the tracking methods above further comprising, after the aseptic procedure, comparing the list of surgical tools used during the aseptic procedure to the initial list to form a list of unused surgical tools.

An optimization method for tool usage in a perioperative environment, the method comprising: prior to initiation of an aseptic procedure, capturing one or more images of a plurality of surgical tools, processing the one or more images to determine an identity of each surgical tool in the plurality of surgical tools, and recording the determined identity of each surgical tool in the plurality of surgical tools to create an initial list of surgical tools; and during the aseptic procedure, repeating the capturing and processing steps substantially in real-time, and recording an identity of each surgical tool removed from the plurality of surgical tools to form a list of surgical tools used during the aseptic procedure.

The optimization method above further comprising, after the aseptic procedure, comparing the list of surgical tools used during the aseptic procedure to the initial list to form a list of unused surgical tools.

Any of the optimization methods above wherein the step of processing the one or more images to determine the identity of each surgical tool in the plurality of surgical tools comprises, comparing the one or more images of the plurality of surgical tools to stored images of a plurality of reference surgical tools; and generating a comparison score for each of the plurality of surgical tools based on the comparing step, wherein the identity of each of the plurality of surgical tools is assigned as an identity of the reference surgical tool having the highest comparison score.

Any of the optimization methods above wherein the step of capturing one or more images of a plurality of surgical tools comprises receiving image data from a camera. The method wherein the camera comprises a 3D camera, a color camera, an IR camera, a UV camera, lidar, or a combination thereof.

Any of the optimization methods above wherein the camera is a body mounted camera, a room mounted camera, or a combination thereof.

Any of the optimization methods above wherein the plurality of surgical tools are arranged on a tray, and the step of capturing the one or more images further comprises capturing an image of a reference tag on the tray, wherein the reference tag provides a reference grid for obtaining dimensions of the plurality of surgical tools, and a 2D code that identifies a digital list of expected surgical tools on the tray.

Any of the optimization methods above wherein the step of processing the one or more images to determine the identity of each surgical tool in the plurality of surgical tools comprises recognizing a reference tag positioned on each of the surgical tools and accessing a reference list database comprising a surgical tool identity related to the reference tag.

A tracking system for a plurality of surgical tools through a use cycle, the system comprising: a server having a processor; at least one optical imaging device in communication with the server; and a memory coupled to the processor and storing processor-readable instructions that, when executed, cause the processor to perform any of the tracking or optimization methods disclosed herein.

The tracking system above wherein the steps of capturing and processing are completed at only specific locations throughout a perioperative environment, such as one or more of an OR, decontamination area, and/or assembly area.

Any of the tracking systems above wherein the steps of capturing and processing are completed substantially continuously as the plurality of surgical tools are transported throughout a perioperative environment.

Any of the tracking systems above wherein the steps of capturing and processing are completed at timed intervals (e.g., every 0.1 seconds, 1 second, 2, seconds, etc.) at only specific locations throughout a perioperative environment or substantially continuously as the plurality of surgical tools are transported throughout a perioperative environment.

Any of the tracking systems above wherein the step of creating the augmented reality information comprises evaluating, via a machine learning algorithm, the captured images; and outputting the augmented reality information such that, in response to a user placing a tray comprising the plurality of surgical tools in a field of view of the user, the identity of the one or more missing surgical tools, a correct location of the one or more missing surgical tools on the tray, or both is perceivable in the field of view of the user.

A non-transitory computer readable recording medium recorded with a program for causing a computer to track a plurality of surgical tools through a use cycle by any of the tracking or optimization methods disclosed herein.

The methods and systems disclosed herein may also find use beyond training, such as medical education, and medical certification. Additionally, the methods and systems of the present disclosure may be configured as games, wherein positive feedback may gain rewards and negative feedback may reduce reward (e.g., score).

As such, while specific embodiments of the disclosure have been described in detail, it should be appreciated by those skilled in the art that various modifications and alternations and applications could be developed in light of the overall teachings of the disclosure. Accordingly, the particular arrangements, systems, apparatuses, and methods disclosed are meant to be illustrative only and not limiting as to the scope of the invention.

1. A method for tracking a plurality of surgical tools through a use cycle, the method comprising:
capturing one or more images of the plurality of surgical tools while in a perioperative environment;
processing the one or more images to determine identities of each surgical tool in the plurality of surgical tools;
recording the determined identities of each surgical tool to form an initial list of identities; and
comparing the initial list of identities of each surgical tool to an expected list of identities.

2. The method of claim 1, wherein the expected list of identities is determined based on a user input value or a fiducial marker found in the one or more images captured by the camera.

3. (canceled)

4. The method of claim 1, wherein the step of processing the one or more images to determine identities of each surgical tool in the plurality of surgical tools comprises:
pattern recognition of a shape and size of each surgical tool, a tag attached to each surgical tool, or a combination of both.

5. (canceled)

6. The method of claim 1, wherein the step of processing the one or more images to determine identities of each surgical tool in the plurality of surgical tools comprises:
comparing the one or more images of the plurality of surgical tools to stored images of a plurality of reference surgical tools; and
generating a comparison score for each of the plurality of surgical tools based on the comparing step,
wherein the identity of each of the plurality of surgical tools is assigned as an identity of the reference surgical tool having the highest comparison score.

7. The method of claim 1, wherein the step of capturing one or more images of the plurality of surgical tools comprises receiving image data from a camera,
wherein the camera comprises a body mounted camera or a room mounted camera.

8. (canceled)

9. The method of claim 1, wherein the plurality of surgical tools are arranged on a tray, and the step of capturing the one or more images further comprises capturing an image of a reference tag on the tray, wherein the reference tag provides:
a reference grid for obtaining dimensions of the plurality of surgical tools, and
a 2D code that identifies the expected list of identities of surgical tools on the tray.

10. The method of claim 1, wherein the step of processing the one or more images to determine the identity of each surgical tool in the plurality of surgical tools comprises:
recognizing a reference tag positioned on each of the surgical tools, and
accessing a reference list database comprising a surgical tool identity related to the reference tag.

11. The method of claim 1, wherein the steps of capturing, processing, and recording are completed at more than one location in the perioperative environment, wherein the perioperative environment includes at least an operating room, a decontamination area, and an assembly area.

12. The method of claim 11, wherein the steps of capturing, processing, and recording are completed at each of the operating room, the decontamination area, and the assembly area.

13. The method of claim 1, wherein the steps of capturing, processing, and recording are completed substantially continuously as the plurality of surgical tools are transported throughout the perioperative environment.

14. The method of claim 1, further comprising:
based on the comparing step, determining one or more surgical tools missing from the plurality of surgical tools, one or more surgical tools added to the plurality of surgical tools, or a combination of both.

15. The method of claim 1, further comprising:
based on the comparing step, determining one or more surgical tools missing from the plurality of surgical tools, one or more surgical tools added to the plurality of surgical tools, or a combination of both;
creating an augmented reality information comprising an identity of each of the one or more surgical tools missing from the plurality of surgical too, added to the plurality of surgical too, or a combination of both; and
outputting the augmented reality information in a field of view of a user.

16. The method of claim 1, wherein the step of capturing an image of the plurality of surgical tools comprises capturing an image of an assembled tray comprising the plurality of surgical tools.

17. The method of claim 16, further comprising:
creating an augmented reality information that relates to one or more missing surgical tools from the assembled tray by evaluating, via a machine learning algorithm, the image captured by the camera of the assembled tray; and
outputting the augmented reality information such that, in response to a user placing the assembled tray in a field of view of the user, an identity and/or correct location of the one or more missing surgical tools is perceivable in the field of view of the user.

18. The method of claim 17, wherein the identity and/or correct location of the one or more missing surgical tools is provided as an overlaid image on the assembled tray in the field of view of the user, wherein the identity and/or correct location of the one or more missing surgical tools is based on a surgeon preference list, a procedure type, or both.

19. (canceled)

20. The method of claim 18, wherein the surgeon preference list includes a list of surgical tools selected by a surgeon or a list of surgical tools determined by the machine learning algorithm.

21. The method of claim 18, wherein the list of surgical tools determined by the machine learning algorithm is identified by a use rate of individual surgical tools in the plurality of surgical tools during a procedure in an aseptic environment.

22. The method of claim 1, wherein the step of capturing one or more images of the plurality of surgical tools is performed in an operating room prior to initiation of an aseptic procedure, and the method further comprises:
during the aseptic procedure, repeating the capturing and processing steps substantially in real-time, and recording an identity of each surgical tool removed from the plurality of surgical tools to form a list of surgical tools used during the aseptic procedure.

23. The method of claim 22, further comprising:
after the aseptic procedure, comparing the list of surgical tools used during the aseptic procedure to the initial list to form a list of unused surgical tools.

24. A method for optimizing tool usage in a perioperative environment, the method comprising:
prior to initiation of an aseptic procedure, capturing one or more images of a plurality of surgical tools, processing the one or more images to determine an identity of each surgical tool in the plurality of surgical tools, and recording the determined identity of each surgical tool in the plurality of surgical tools to create an initial list of surgical tools;
during the aseptic procedure, repeating the capturing and processing steps substantially in real-time, and recording an identity of each surgical tool removed from the plurality of surgical tools to form a list of surgical tools used during the aseptic procedure; and
after the aseptic procedure, comparing the list of surgical tools used during the aseptic procedure to the initial list to form a list of unused surgical tools.

25. (canceled)

26. The method of claim 24, wherein the step of processing the one or more images to determine the identity of each surgical tool in the plurality of surgical tools comprises:
comparing the one or more images of the plurality of surgical tools to stored images of a plurality of reference surgical tools; and
generating a comparison score for each of the plurality of surgical tools based on the comparing step,
wherein the identity of each of the plurality of surgical tools is assigned as an identity of the reference surgical tool having the highest comparison score.

27. The method of claim 26, wherein the step of capturing one or more images of a plurality of surgical tools comprises receiving image data from a camera,
wherein the camera comprises a body mounted camera or a room mounted camera.

28. (canceled)

29. The method of claim 26, wherein the plurality of surgical tools are arranged on a tray, and the step of capturing the one or more images further comprises capturing an image of a reference tag on the tray, wherein the reference tag provides:
a reference grid for obtaining dimensions of the plurality of surgical tools, and
a 2D code that identifies a digital list of expected surgical tools on the tray.

30. The method of claim 24, wherein the step of processing the one or more images to determine the identity of each surgical tool in the plurality of surgical tools comprises:
recognizing a reference tag positioned on each of the surgical tools, and
accessing a reference list database comprising a surgical tool identity related to the reference tag.
